(19) United States
(12) Patent Application Publication
(10) Pub. No.: US 2023/0190700 A1
(43) Pub. Date: Jun. 22, 2023

(54) THEAFLAVIN COMPOSITION CAPABLE OF PROMOTING WOUND HEALING AS WELL AS PREPARATION METHOD AND USE THEREOF

(71) Applicants: ANHUI AGRICULTURAL UNIVERSITY, Hefei (CN); ANHUI RUNCHENG BIOLOGICAL TECHNOLOGY CO., LTD, Hefei (CN)

(72) Inventors: Yan XU, Hefei (CN); Xiaobing CHEN, Hefei (CN); Xu DONG, Hefei (CN); Zenghui LIU, Hefei (CN); Jiayue JIANG, Hefei (CN); Xiaochun WAN, Hefei (CN); Daxiang LI, Hefei (CN); Ying WANG, Hefei (CN); Luwei ZHU, Hefei (CN)

(21) Appl. No.: 18/173,230

(22) Filed: Feb. 23, 2023

Related U.S. Application Data
(63) Continuation of application No. PCT/CN2022/105315, filed on Jul. 13, 2022.

(30) Foreign Application Priority Data
Jul. 26, 2021 (CN) 202110842893.4

(51) Int. Cl. A61K 31/352; A61K 47/42; A61K 47/36; A61K 47/32; A61K 9/06; A61K 47/10; A61K 47/18; A61P 17/02

(52) U.S. Cl. CPC A61K 31/352; A61K 47/42; A61K 47/36; A61K 47/32; A61K 9/06; A61K 47/10; A61K 47/18; A61P 17/02

(57) ABSTRACT

The present disclosure provides a theaflavin composition capable of promoting wound healing as well as a preparation method and use thereof. The theaflavin composition comprises theaflavin 3′-digallate or crude theaflavins as the main component; when the theaflavin composition comprises the theaflavin-3-3′-digallate as the main component, it further comprises component A or component B, or further comprises both component A and component B; when the theaflavin composition comprises the crude theaflavins as the main component, it further comprises component B, in which component A comprises sodium acetate, chitosan, and gelatin, and component B comprises glycerin, propylene glycol, triethanolamine, and Carbomer 940. The theaflavin composition provided by the present disclosure can significantly promote wound healing of diabetic mice.

### TECHNICAL FIELD

The present disclosure belongs to the technical field of medicines, particularly relates to a theaflavin composition capable of promoting wound healing as well as a preparation method and use thereof.

### BACKGROUND ART

Wound healing is a complicated physiological process in which damaged tissues are restored to make them complete. With the improvement of people's living level, the morbidity of diabetes increases year by year. Diabetic chronic wound, which is one of diabetic complications, is difficult to heal. Of the patients with diabetic chronic wounds, 85% of patients have been subjected to amputation surgery. Diabetic wounds bring economic and healthy dual burdens for patients. Therefore, ideal wound treatment drugs can not only promote wound healing, thereby alleviating patients' pains, but also reduce economic burden of patients.

Theaflavin-3-3′-digallate, namely TFDG, is one of main components of theaflavins in black tea. The crude theaflavins (TFs) have antioxidant, anti-tumor, antibacterial, anti-virus, and anti-inflammatory activities, and can prevent cardiovascular diseases. The isolated and purified theaflavin monomers (theaflavin, theaflavin-3-gallate, theaflavin-3′-gallate, or theaflavin-3-3′-digallate) also have anti-inflammatory and antioxidant efficacies and the like. However, theaflavins are not well applied in the aspect of drug application currently. Thus the present disclosure provides a new direction in the aspect of wound healing.

Nano-materials have the advantages of improving the stability of drug molecules, increasing the bioavailability of drug molecules, improving the poor dissolution and low treatment efficiency of traditional drugs, and the like; gel materials have good water absorbing quality, good moisture retention, and favorable biocompatibility. By combining the advantages of these two materials, the present disclosure invents a novel biological macromolecule polymeric material.

### SUMMARY OF THE INVENTION

The present disclosure aims to provide several theaflavin compositions capable of promoting wound healing, and is intended to solve the problems in the prior art that theaflavin is not well utilized in the aspect of drug application.

In order to realize the above objective, the present disclosure provides the following technical solutions.

Provided firstly is a theaflavin composition capable of promoting wound healing, which comprises theaflavin-3-3′-digallate or crude theaflavins as the main component;

when the theaflavin composition capable of promoting wound healing comprises the theaflavin 3′-digallate as the main component, it further comprises component A or component B, or further comprises both component A and component B;

when the theaflavin composition capable of promoting wound healing comprises the crude theaflavins as the main component, it further comprises component B;

the component A comprises sodium acetate, chitosan, and gelatin;

the component B comprises glycerin, propylene glycol, triethanolamine, and Carbomer 940.

As a further embodiment of the present disclosure, when the theaflavin composition capable of promoting wound healing comprises the theaflavin-3-3′-digallate as the main component and further comprises both component A and component B, the theaflavin-3-3′-digallate, sodium acetate, chitosan, gelatin, glycerin, propylene glycol, triethanolamine, and Carbomer 940 are contained in the theaflavin composition at a mass ratio of 5-30:16-820:1-30:1-30:25-75:20-42:1-11:8-80.

Provided also is a preparation method of the theaflavin composition capable of promoting wound healing described above, which comprises the following steps:

preparing a sodium acetate buffer solution;

preparing a gelatin buffer solution and a chitosan buffer solution, respectively, with the sodium acetate buffer solution, and filtering the same;

dissolving the theaflavin-3-3′-digallate into the chitosan buffer solution to obtain a mixed solution;

dropwise adding the mixed solution into the gelatin buffer solution, stirring, centrifuging, and collecting the precipitate;

redissolving the precipitate into water by 1/10 of the original volume, adding glycerin, propylene glycol, and Carbomer 940 according to a raw material formula, mixing uniformly, and standing to obtain a nano gel solution; and

adding triethanolamine into the nano gel solution, and stirring to obtain the theaflavin composition capable of promoting wound healing.

As a further embodiment of the present disclosure, the gelatin buffer solution and the chitosan buffer solution are filtered using a 0.45 μm aquo-system filter membrane.

As a further embodiment of the present disclosure, the theaflavin-3-3′-digallate is dissolved into the chitosan buffer solution with the aid of ultrasound, and the precipitate is redissolved into water with the aid of ultrasound.

As a further embodiment of the present disclosure, the centrifugation treatment is carried out for 15-60 min at the temperature of 2-6° C. at the rotation speed of 3000-5000 r/min.

As a further embodiment of the present disclosure, the magnetic stirring treatment is specifically conducted for 20-50 min at the rotation speed of 250-350 r/min.

As a further embodiment of the present disclosure, the mixed solution is dropwise added at the speed of 0.5-2 mL/min.

As a further embodiment of the present disclosure, the nano gel solution stands for 8-24 hours.

As another embodiment of the present disclosure, provided is use of a theaflavin composition capable of promoting wound healing in preparing a drug for accelerating the wound healing, wherein the theaflavin composition capable of promoting wound healing is the above theaflavin composition capable of promoting wound healing, or is prepared by the above preparation method of the theaflavin composition capable of promoting wound healing.

Beneficial Effects

The theaflavin composition capable of promoting wound healing provided by the present disclosure is prepared by using the theaflavin-3-3′-digallate or crude theaflavins, sodium acetate, chitosan, gelatin, glycerin, propylene glycol, triethanolamine, and Carbomer 940 as raw materials, and the obtained theaflavin composition capable of promoting wound healing can effectively promote the synthesis of collagen proteins, reduce the infiltration of inflammatory cells, accelerate the differentiation of regenerative cells and the formation of blood vessels, and significantly promote the wound healing of diabetes mice, and has great potential application value for wound healing.

### BRIEF DESCRIPTION OF THE DRAWINGS

FIG. 1 is a transmission electron microscope image of nano particles in theaflavin compositions capable of promoting wound healing provided in Examples 1-2;

FIG. 2 is a graph showing the wound healing of mice in different treatment groups provided in Examples of the present disclosure;

FIG. 3 is a bar graph showing wound healing rates of mice in different treatment groups provided in Examples of the present disclosure;

FIG. 4 is a graph showing H&E staining results of skin wounds of mice in different treatment groups on Day 12 provided in Examples of the present disclosure;

FIG. 5 is a graph showing Masson staining results of skin wounds of mice in different treatment groups on Day 12 provided in Examples of the present disclosure;

FIG. 6 is a graph showing immunohistochemical (IHC) straining results of skin wounds of mice in different treatment groups on Day 12 provided in Examples of the present disclosure.

### DETAILED DESCRIPTION OF THE INVENTION

To make the purpose, technical solution, and advantages of the present disclosure more clear, the present disclosure will be further described in detail in combination with Drawings and Examples. It should be understood that specific examples described herein are only for explaining the present disclosure but not limiting the present disclosure.

In order to solve the problem in the prior art that theaflavin has not been well utilized in the aspect of drug utilization, the present disclosure provides several theaflavin compositions capable of promoting wound healing, which are prepared by using the theaflavin-3-3′-digallate or crude theaflavins, sodium acetate, chitosan, gelatin, glycerin, propylene glycol, triethanolamine, and Carbomer 940 as raw materials, and the obtained theaflavin compositions capable of promoting wound healing can effectively promote the synthesis of collagen proteins, reduce the infiltration of inflammatory cells, accelerate the differentiation of regenerative cells and the formation of blood vessels, and significantly promote the wound healing of diabetes mice, and has great potential application value for wound healing.

Without intent to further limit the scope of the present disclosure, examples in some embodiments of the present disclosure are given below.

In addition, it is noted that numerical values given in the following examples are as accurate as possible, and however, those skilled in the art will appreciate that due to inevitable measurement errors and experimental operation problems, each number should be understood as an approximate number, but not an absolutely accurate number. For example, due to errors of weighing machines, it should be understood that the content values of raw materials for preparing nano particle compositions in examples may have an error of ±1 or ±2.

### Example 1

This example provides a theaflavin composition 1 capable of promoting wound healing, which comprises the following main components: theaflavin-3-3′-digallate, sodium acetate, chitosan, gelatin, glycerin, propylene glycol, triethanolamine, and Carbomer 940, at a mass ratio of 10:164:5:5:19:10:1:1. The theaflavin composition 1 capable of promoting wound healing was prepared according to the following steps:

1, anhydrous sodium acetate was dissolved into ultra-pure water to prepare 0.2 mol/L sodium acetate solution, and then the pH of the sodium acetate solution was adjusted to 5.4 using hydrochloric acid or sodium hydroxide, so as to obtain a sodium acetate buffer solution; 2, chitosan and gelatin were respectively dissolved into the sodium acetate buffer solution to prepare 0.1% gelatin buffer solution and 0.1% chitosan buffer solution, both of which were then filtered with a 0.45 μm aquo-system filter membrane; 3, 50 mg of theaflavin-3-3′-digallate was ultrasonically dissolved into 25 mL of 0.1% chitosan buffer solution until completely dissolved, and the obtained mixed solution was then slowly dropwise added into 25 mL of 0.1% gelatin buffer solution at a speed of 1 mL/min, and mixed uniformly, followed by magnetically stirring for 30 min at 25° C. at the rotation speed of 300 r/min, and then a nano suspension was obtained after completely mixing;

4, the suspension was centrifuged for 20 minutes at 4° C. at 5000 r/min, and then the precipitate was collected;

5, the precipitate was redissolved into water by 1/10 of the original volume with the aid of ultrasound, and subsequently glycerin, propylene glycol, and Carbomer 940 were added according to a raw material formula, followed by standing for 12 hours to obtain a nano gel solution; and

6, triethanolamine was added into the nano gel solution, and stirred to obtain the theaflavin composition 1 capable of promoting wound healing.

### Example 2

This example provides a theaflavin composition 2 capable of promoting wound healing, which comprises the following main components: theaflavin-3-3′-digallate, sodium acetate, chitosan, and gelatin, at a mass ratio of 10:164:5:5. The theaflavin composition 2 capable of promoting wound healing was prepared according to the following steps:

1, anhydrous sodium acetate was dissolved into ultra-pure water to prepare 0.2 mol/L sodium acetate solution, and then the pH of the sodium acetate solution was adjusted to 5.4 using hydrochloric acid or sodium hydroxide, so as to obtain a sodium acetate buffer solution;

2, chitosan and gelatin were respectively dissolved into the sodium acetate buffer solution to prepare 0.1% gelatin buffer solution and 0.1% chitosan buffer solution, both of which were then filtered with a 0.45 μm aquo-system filter membrane;

3, 50 mg of theaflavin-3-3′-digallate was ultrasonically dissolved into 25 mL of 0.1% chitosan buffer solution until completely dissolved, and then the obtained mixed solution was slowly dropwise added into 25 mL of 0.1% gelatin buffer solution at a speed of 1 mL/min, and mixed uniformly, followed by magnetically stirring for 30 min at 25° C. at the rotation speed of 300 r/min, and then a nano suspension was obtained after completely mixing;

4, the suspension was centrifuged for 20 minutes at 4° C. at 5000 r/min, and then the precipitate (nano particles) was collected; and

5, the precipitate was redissolved into water by 1/10 of the original volume with the aid of ultrasound to obtain the theaflavin composition 2 capable of promoting wound healing.

### Example 3

This example provides a theaflavin composition 3 capable of promoting wound healing, which comprises the following main components: theaflavin-3-3′-digallate, glycerin, propylene glycol, triethanolamine, and Carbomer 940, at a mass ratio of 10:19:10:1:1.

The theaflavin composition 3 capable of promoting wound healing was prepared according to the following steps:

1, 100 mg of theaflavin-3-3′-digallate was ultrasonically dissolved into 10 mL of water;

2, glycerin, propylene glycol, and Carbomer 940 were added according to a raw material formula and allowed to stand for a certain period of time to obtain a gel solution; and

3, triethanolamine was added into the gel solution, and stirred to obtain the theaflavin composition 3 capable of promoting wound healing.

### Example 4

This example provides a theaflavin composition 4 capable of promoting wound healing, which comprises the following main components: crude theaflavins (mainly including theaflavin (TF, 10±5%), theaflavin-3-gallate (TF-3-G, 15±5%), theaflavin-3′-gallate (TF-3′-G, 5±5%), and theaflavin-3-3′-digallate (TF-3-3′-G, 15±5%)), glycerin, propylene glycol, triethanolamine, and Carbomer 940, at a mass ratio of 20:19:10:1:1. The theaflavin composition 4 capable of promoting wound healing was prepared according to the following steps:

1, 200 mg of crude theaflavins was ultrasonically dissolved into 10 mL of water;

2, glycerin, propylene glycol, and Carbomer 940 were added according to a raw material formula and allowed to stand for a certain period of time to obtain a gel solution; and

3, triethanolamine was added into the gel solution, and stirred to obtain the theaflavin composition 4 capable of promoting wound healing.

### Example 5

This example provides a composition 5, which comprises the main components: glycerin, propylene glycol, triethanolamine, and Carbomer 940, at a mass ratio of 19:10:1:1. The composition 5 was prepared according to the following steps:

1, glycerin, propylene glycol, and Carbomer 940 were added into 10 mL of water according to a raw material formula and allowed to stand for a certain period of time to obtain a gel solution; and

2, triethanolamine was added into the gel solution, and stirred to obtain the composition 5.

### Example 6

This example provides a theaflavin composition 6 capable of promoting wound healing, which comprises the following main components: theaflavin-3-3′-digallate, sodium acetate, chitosan, gelatin, glycerin, propylene glycol, triethanolamine, and Carbomer 940, at a mass ratio of 5:16:1:1:13:10:1:8. The theaflavin composition 6 capable of promoting wound healing was prepared according to the following steps:

1, anhydrous sodium acetate was dissolved into ultra-pure water to prepare 0.01 mol/L sodium acetate solution, and then the pH of the sodium acetate solution was adjusted to 5.0 using hydrochloric acid or sodium hydroxide, so as to obtain a sodium acetate buffer solution;

2, chitosan and gelatin were respectively dissolved into the sodium acetate buffer solution to prepare 0.01% gelatin buffer solution and 0.01% chitosan buffer solution, both of which were then filtered with a 0.45 μm aquo-system filter membrane;

3, 50 mg of theaflavin-3-3′-digallate was ultrasonically dissolved into 25 mL of 0.01% chitosan buffer solution until completely dissolved, and then the obtained mixed solution was slowly dropwise added into 25 mL of 0.01% gelatin buffer solution at a speed of 1 mL/min and mixed uniformly, followed by magnetically stirring for 30 min at 25° C. at the rotation speed of 300 r/min, and then a nano suspension was obtained after completely mixing;

4, the suspension was centrifuged for 20 minutes at 4° C. at 5000 r/min, and then the precipitate was collected;

5, the precipitate was redissolved into water by 1/10 of the original volume with the aid of ultrasound, and subsequently glycerin, propylene glycol, and Carbomer 940 were added according to a raw material formula, followed by standing for 12 hours to obtain a nano gel solution; and

6, triethanolamine was added into the nano gel solution, and stirred to obtain the theaflavin composition 6 capable of promoting wound healing.

### Example 7

This example provides a theaflavin composition 7 capable of promoting wound healing, which comprises the following main components: theaflavin-3-3′-digallate, sodium acetate, chitosan, gelatin, glycerin, propylene glycol, triethanolamine, and Carbomer 940, at a mass ratio of 30:820:30:30:75:42:11:80. The theaflavin composition 7 capable of promoting wound healing was prepared according to the following steps:

1, anhydrous sodium acetate was dissolved into ultra-pure water to prepare 0.05 mol/L sodium acetate solution, and then the pH of the sodium acetate solution was adjusted to 5.4 using hydrochloric acid or sodium hydroxide, so as to obtain a sodium acetate buffer solution;

2, chitosan and gelatin were respectively dissolved into the sodium acetate buffer solution to prepare 0.03% gelatin buffer solution and 0.03% chitosan buffer solution, both of which were then filtered with a 0.45 μm aquo-system filter membrane;

3, 50 mg of theaflavin-3-3′-digallate was ultrasonically dissolved into 25 mL of 0.03% chitosan buffer solution until completely dissolved, and then the obtained mixed solution was slowly dropwise added into 25 mL of 0.03% gelatin buffer solution at a speed of 1 mL/min and mixed uniformly, followed by magnetically stirring for 30 min at 25° C. at the rotation speed of 300 r/min, and then a nano suspension was obtained after complete mixing;

4, the suspension was centrifuged for 20 minutes at 4±2° C. at 5000 r/min, and then the precipitate was collected;

5, the precipitate was redissolved into water by 1/10 of the original volume with the aid of ultrasound, and subsequently glycerin, propylene glycol, and Carbomer 940 were added according to a raw material formula, followed by standing for 12 hours to obtain a nano gel solution; and

6, triethanolamine was added into the nano gel solution, and stirred to obtain the theaflavin composition 7 capable of promoting wound healing

### Example 8

The preparation method in this example is substantially the same as that in Example 1, except that the centrifugation treatment was conducted for 15 min at 2° C. at the rotation speed of 3000 r/min.

### Example 9

The preparation method in this example is substantially the same as that in Example 1, except that the centrifugation treatment was conducted for 30 min at 6° C. at the rotation speed of 5000 r/min.

### Example 10

The preparation method in this example is substantially the same as that in Example 1, except that the magnetic stirring treatment was conducted for 20 min at the rotation speed of 250 r/min.

### Example 11

The preparation method in this example is substantially the same as that in Example 1, except that the magnetic stirring treatment was conducted for 50 min at the rotation speed of 350 r/min

### Example 12

The preparation method in this example is substantially the same as that in Example 1, except that the mixed solution was dropwise added at a speed of 0.5 mL/min.

### Example 13

The preparation method in this example is substantially the same as that in Example 1, except that the mixed solution was dropwise added at a speed of 2 mL/min.

### Example 14

The preparation method in this example is substantially the same as that in Example 1, except that the nano gel solution was allowed to stand for 8 hours.

### Example 15

The preparation method in this example is substantially the same as that in Example 1, except that the nano gel solution was allowed to stand for 24 hours.

Experiment Verification

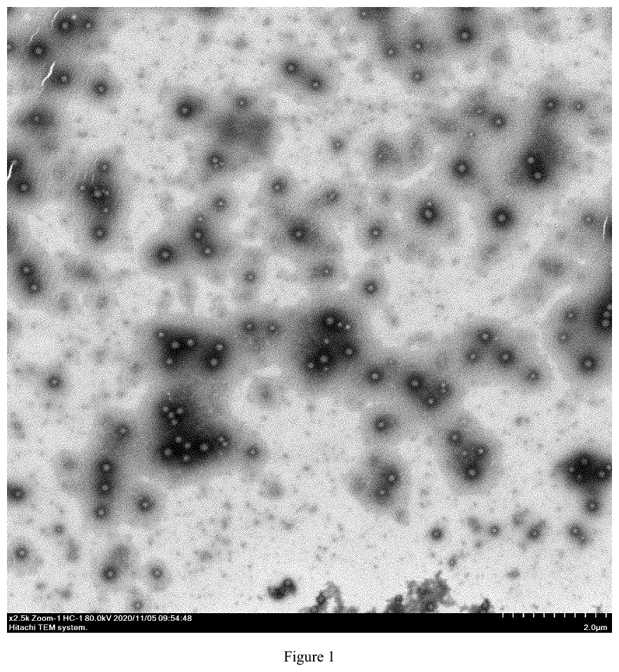

The theaflavin composition capable of promoting wound healing prepared in Example 2 were subjected to transmission electron microscope scanning for observing its morphological structure. The result was shown in FIG. 1, which showed that the theaflavin composition capable of promoting wound healing had no obvious aggregation and was uniform in size. The theaflavin compositions capable of promoting the wound healing prepared in Examples 1-5 were subjected to animal experiments, and the specific method was as follows:

(1) Construction of an animal model of diabetes mellitus

60 male ICR mice with a body weight of 30±2 g, eight-week-old, were provided by Beijing Weitong Lihua Co., Ltd. These mice were placed in a SPF-grade animal room with an ambient temperature of 23±2° C. and a relative humidity of 50±10%, and then adaptively fed for one week under the conditions of 12-hour light illumination and 12-hour darkness. The night before modeling, the mice were fasted but allowed to drink water for 12 hours, and then the body weights and fasting blood glucose were measured. For model and test groups, single intraperitoneal injection of streptozotocin (STZ) solution (120 mg/kg) was conducted based on the body weight; for blank control group, citric acid/sodium citrate buffer solution was injected based on the body weight, and then the mice were stably fed for 5 days. The night before experiment, the mice were fasted but allowed to drink water for 12 hours, then blood glucose was measured, and the mice with a blood glucose value of more than 11.1 mmol/L were deemed as diabetes mice. The diabetes mice were grouped at random and adaptively fed for three days before skin wound modeling.

(2) Construction of Skin Wound Model

The mice were divided into 8 groups (n=7), which were respectively as follows: blank control group C (Control), model group M (Model), positive drug group R (Recombinant bovine basic fibroblast growth factor gel, which was purchased from Zhuhai Yisheng Biopharmaceutical Co., Ltd), non-loaded gel group E (empty gel, i.e., the composition 5 prepared in Example 5), crude theaflavins gel group T (TFs Gel, i.e., the theaflavin composition 4 capable of promoting wound healing prepared in Example 4), theaflavin-3-3′-digallate gel group DGG (TFDG Gel, i.e., the theaflavin composition 3 capable of promoting wound healing prepared in Example 3), theaflavin-3-3′-digallate nano particle group DGN (TFDG NPS, i.e., the theaflavin composition 2 capable of promoting wound healing prepared in Example 2), theaflavin-3-3′-digallate nano gel group DGNG (TFDG NPS Gel, i.e., the theaflavin composition 1 capable of promoting wound healing prepared in Example 1).

Each of the above groups was established as follows.

Blank control group: mice in this group were normal mice with 0.9% normal saline smeared at the wound.

Model group: mice in this group were diabetic model mice with 0.9% normal saline smeared at the wound.

Positive drug group: mice in this group were diabetic model mice with 0.9% recombinant bovine basic fibroblast growth factor gel smeared at the wound.

Non-loaded gel group: mice in this group were diabetic model mice with the composition 5 obtained in Example 5 smeared at the wound.

Crude theaflavins gel group: mice in this group were diabetic model mice with the theaflavin composition 4 capable of promoting wound healing obtained in Example 4 smeared at the wound. Theaflavin-3-3′-digallate gel group: mice in this group were diabetic model mice with the theaflavin composition 3 capable of promoting wound healing obtained in Example 3 smeared at the wound. Theaflavin-3-3′-digallate nano particle group: mice in this group were diabetic model mice with the theaflavin composition 2 capable of promoting wound healing obtained in Example 2 smeared at the wound Theaflavin-3-3′-digallate nano gel group: mice in this group were diabetic model mice with the theaflavin composition 1 capable of promoting wound healing obtained in Example 1 smeared at the wound.

Each mouse was anesthetized by intraperitoneal injection of 4% chloral hydrate (0.01 mL/g), the hairs on the back were removed with hair removal ointment, and two symmetrical round wounds with a diameter of 6 mm were created on the back of the mouse with a hole punch. The mice were fed separately. The initial wound area was photographed with a digital camera, which was set as Day 0. Drug was administered once a day for 12 days in total, photos were taken every other day, and then the wound healing rate was calculated and the wound healing condition was recorded. On Day 12, the skins at the wounds of mice in each group were taken and soaked in formalin for 7 days, and then subjected to paraffin embedding, sectioning, and staining.

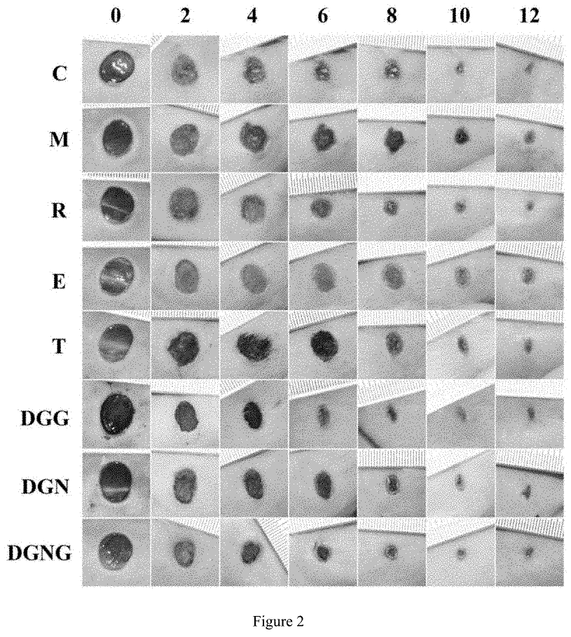

FIG. 2 is a graph showing skin wounds of mice in different treatment groups on different days. In the figure, C represents the blank control group, M represents the model group, R represents the positive drug group, E represents the non-loaded gel group, T represents the crude theaflavins gel group, DGG represents the theaflavin-3-3′-digallate gel group, DNG represents the theaflavin-3-3 ‘-digallate nano particle group, and DGNG represents the theaflavin-3-3’-digallate nano gel group. It can be seen from FIG. 2 that with the increase of time, for each group of mice, the wounds gradually healed, and the areas of the wounds were gradually reduced. Among others, on Day 12, the wound healing areas of mice in the model group M and the empty gel group E were relatively small, and the wound healing areas of mice in the administration groups R, DGG, DGN, and DGNG were all larger than those of the model group M.

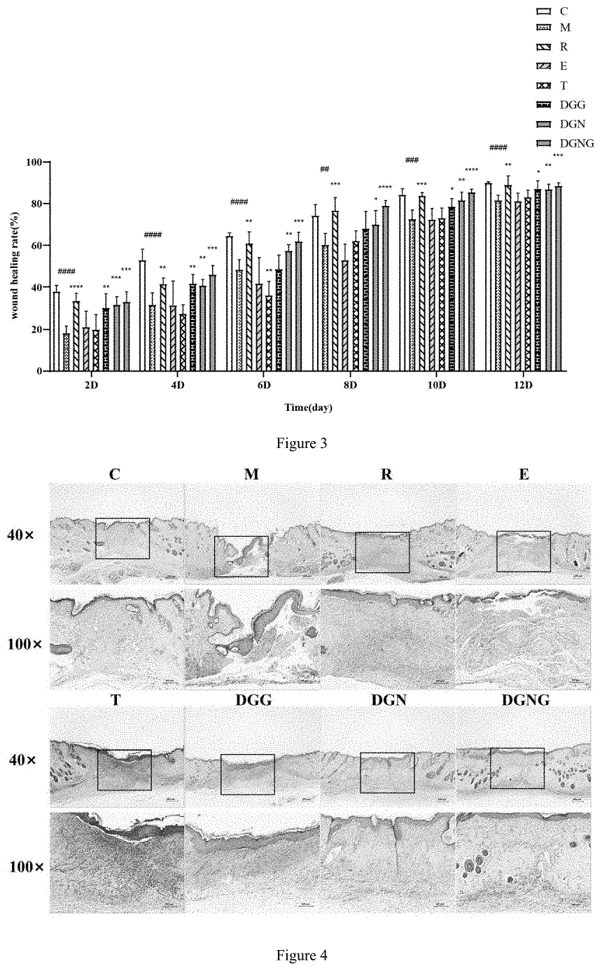

In FIG. 3, data are shown in mean±SEM, #represents that there is significant difference compared with the blank control group C (P<0.05), and * represents that there is significant difference compared with the model group M (P<0.05).

FIG. 2 shows the wound conditions recorded during the test. When compared with the model group, the wound areas of the five administration groups, that is, the positive drug group, the crude theaflavins gel group, the theaflavin-3-3′-digallate gel group, the theaflavin-3-3′-digallate nano particle group, and the theaflavin-3-3′-digallate nano gel group, were obviously reduced, and the skins of the five administration groups were more elastic. For the model group, the wound periphery had serious inflammation, and the skin around the wound tended to form blood scabs; and for the five administration groups, the wound periphery had no inflammation. Among the administration groups, the blood scabs of the nano particle group and of the nano gel particle group were relatively hard and dry, the skin around the scabs was dry and clean, and the scabs were more easier to fall off, which was conducive to wound healing; the crude theaflavins gel group and the theaflavin-3-3′-digallate gel group had relatively thick and viscous scabs. Starting from the second day, there was a significant difference in the wound healing rate between the blank control group and the model group (P<0.01), indicating that diabetes has a great influence on wound healing. On Day 12, there was significant difference in the wound healing degree between each of the five administration groups and the model group (P<0.001), proving that the nano gel composition provided by the examples of the present disclosure can accelerate the wound healing of the diabetes mice.

FIG. 4 is a graph showing H&E staining of skin wounds of mice on day 12, with magnification of 40 and 100 folds respectively. It can be seen from FIG. 4 that the wound healing of mice in the crude theaflavins gel group, the theaflavin-3-3′-digallate gel group, the theaflavin-3-3′-digallate nano particle group, and the theaflavin-3-3′-digallate nano gel group was obviously superior to that of the model group and of the empty gel group. For mice in the blank control group (non-diabetes group), the wounds almost healed on day 12, and the wound keratinocytes were orderly arranged so as to form new epidermis; fibroblasts migrated into the wounds to connect the wounds and were transformed into myofibroblasts which formed granulation tissues. Compared with the blank control group, the epidermis of mice in the model group had not been completely regenerated yet, in which intrinsic granulation tissues were less, a large number of inflammatory cells were infiltrated, and regenerative blood vessels are less formed. Some new blood vessels and epidermis were formed in the wounds of mice in the empty gel group, and however, the newly formed granulation tissues were loosely arranged. For the positive drug group, the crude theaflavins gel group, the theaflavin-3-3′-digallate gel group, the theaflavin-3-3′-digallate nano particle group, and the theaflavin-3-3′-digallate nano gel group, keratinocytes were regenerated and fibroblasts were orderly arranged and formed new blood vessels, wherein the newly formed skins of mice in the theaflavin-3-3′-digallate nano gel group grew faster and were superior to those of other groups.

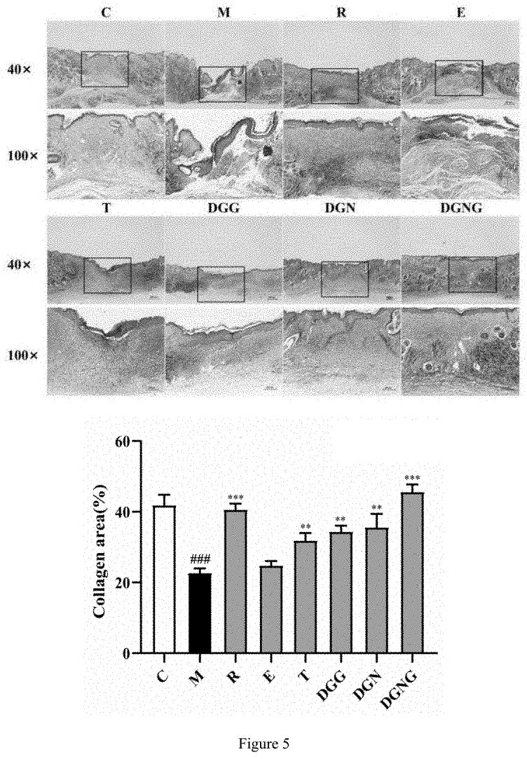

FIG. 5 is a graph showing Masson staining of skin wounds of mice on Day 12 for observing the deposition of collagen fibers. During the wound healing, collagenous fibers play an important role. Migration of fibroblasts to connect the wounds and in turn heal the wounds is conducted by regulating the quantity of collagen proteins. The accumulation of collagen proteins promotes the formation of granulation tissues and fiber cells. On Day 12, the deposition amounts of collagen fibers in the positive drug group, the crude theaflavins gel group, the theaflavin-3-3′-digallate gel group, the theaflavin-3-3′-digallate nano particle group, and the theaflavin-3-3′-digallate nano gel group were significantly higher than those of the model group and of the non-loaded gel group (P<0.001).

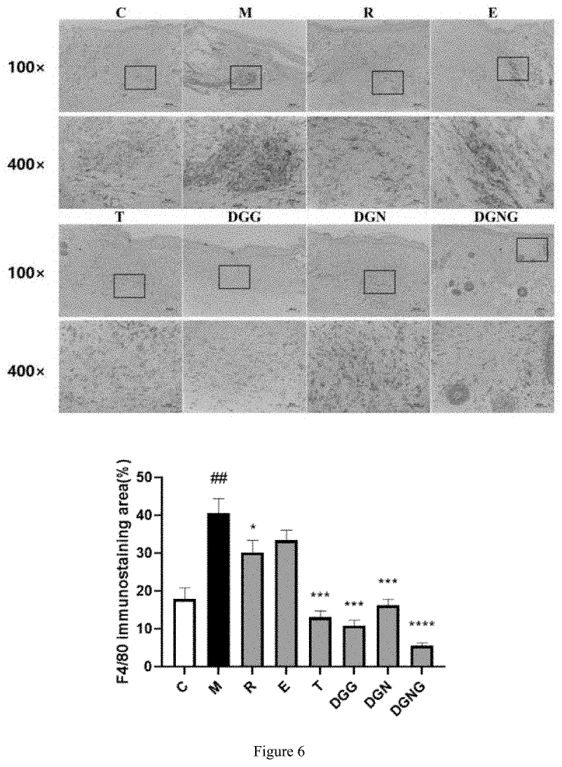

In FIG. 6, (A) represents an immunohistochemical staining graph of wounds of mice on Day 12, in which macrophages (F4/80) in the skin wound were in brownish yellow, and the nuclei were stained by hematoxylin; the magnifications were 100 folds and 400 folds, respectively; (B) represents a statistical chart showing the percentage of macrophages in the total area. #represents that there is a significant difference compared with the control group (P<0.05). * represents that there is a significant difference compared with the model group (P<0.05).

As can be seen from FIG. 6, the inflammatory cells of the blank control group were significantly less than those of the model group (P<0.5). The inflammatory cells of the crude theaflavins gel group, of the theaflavin-3-3′-digallate gel group, of the theaflavin-3-3 ‘-digallate nano particle group, and of the theaflavin-3-3’-digallate nano gel group were significantly less than those of the model group and of the non-loaded gel group (P<0.5). In addition, it was found that the inflammatory cells of the positive drug group were significantly more than those of the crude theaflavins gel group, of the theaflavin-3-3′-digallate gel group, of the theaflavin-3-3′-digallate nano particle group, and of the theaflavin-3-3′-digallate nano gel group. The results show that the theaflavin compositions prepared by the examples of the present application can significantly reduce the quantity of macrophages (F4/80) in the wounds of diabetes mice.

In summary, the present theaflavin composition capable of promoting wound healing prepared by using theaflavin-3-3′-digallate or crude theaflavins, sodium acetate, chitosan, gelatin, glycerin, propylene glycol, Carbomer 940, and triethanolamine as raw materials can effectively promote the synthesis of collagen proteins at the skin wound, reduce the infiltration of inflammatory cells, accelerate the differentiation of regenerative cells and the formation of blood vessels, and significantly promote the wound healing of diabetes mice, and has a great potential application value in the aspect of wound healing.

The above examples merely express several embodiments of the present disclosure, their descriptions are specific and detailed, but cannot be understood as limiting the scope of the present invention patent. It should be noted that for persons of ordinary skill in the art, several deformations and improvements can also be made without departing from the concept of the present disclosure, which are all included within the protective scope of the present disclosure. Therefore, the protection scope of the invention shall be based on the content of its claims.

The foregoing just presents better examples of the present disclosure, and is not intended to limit the invention. Any modification, equivalent substitution, improvement, etc. made within the spirit and principles of the invention shall be included in the scope of protection of the invention.

1-11. (canceled)

12. A theaflavin composition capable of promoting wound healing, which comprises theaflavin-3-3′-digallate, sodium acetate, chitosan, and gelatin;
wherein theaflavin-3-3′-digallate, sodium acetate, chitosan, and gelatin are contained in the theaflavin composition at a mass ratio of 10:164:5:5; and
wherein the theaflavin composition capable of promoting wound healing is prepared by a method comprising the steps of:
(1) preparing 0.2 mol/L sodium acetate solution by dissolving anhydrous sodium acetate in ultrapure water, and adjusting the pH value of the sodium acetate solution to 5.4 with hydrochloric acid or sodium hydroxide, to obtain a sodium acetate buffer solution;
(2) preparing 0.1% chitosan buffer solution and 0.1% gelatin buffer solution by dissolving chitosan and gelatin into the sodium acetate buffer solution, respectively, and filtering the same with a 0.45 μm aquo-system filter membrane;
(3) dissolving 50 mg of the theaflavin-3-3′-digallate into 25 mL of the 0.1% chitosan buffer solution with the aid of ultrasound, and upon complete dissolution, slowly dropwise adding the solution into 25 mL of the 0.1% gelatin buffer solution at a speed of 1 mL/min;
mixing uniformly, and magnetically stirring at 300 r/min at 25° C. for 30 min until well mixed to obtain a nano suspension;
(4) centrifuging the nano suspension at 5000 r/min at 4° C. for 20 min, and then collecting the precipitate; and
(5) redissolving the precipitate by 1/10 of the original volume with the aid of ultrasound.

13. A theaflavin composition capable of promoting wound healing, which comprises the theaflavin-3-3′-digallate as the active component and further comprises sodium acetate, chitosan, gelatin, glycerin, propylene glycol, triethanolamine, and Carbomer 940;
wherein the theaflavin-3-3′-digallate, sodium acetate, chitosan, gelatin, glycerin, propylene glycol, triethanolamine, and Carbomer 940 are contained in the theaflavin composition at a mass ratio of 5-30:16-820:1-30:1-30:25-75:20-42:1-11:8-80; and,
wherein the theaflavin composition capable of promoting wound healing is prepared by a method comprising the steps of:
preparing a sodium acetate buffer solution;
preparing a gelatin buffer solution and a chitosan buffer solution, respectively, with the sodium acetate buffer solution, and filtering the same;
dissolving theaflavin-3-3′-digallate into the chitosan buffer solution to obtain a mixed solution;
dropwise adding the mixed solution into the gelatin buffer solution, stirring, centrifuging, and collecting the precipitate;
redissolving the precipitate into water by 1/10 of the original volume, adding glycerin, propylene glycol, and Carbomer 940 according to a raw material formula, mixing uniformly, and standing to obtain a nano gel solution; and
adding triethanolamine into the nano gel solution, and stirring to obtain the theaflavin composition capable of promoting wound healing;
wherein the gelatin buffer solution and the chitosan buffer solution are filtered using a 0.45 μm aquo-system filter membrane;
wherein the theaflavin-3-3′-digallate is dissolved into the chitosan buffer solution with the aid of ultrasound, and the precipitate is redissolved into water with the aid of ultrasound;
wherein the centrifugation treatment is carried out for 15-60 min at the temperature of 2-6° C. at the rotation speed of 3000-5000 r/min;
wherein the magnetic stirring treatment is specifically conducted for 20-50 min at the rotation speed of 250-350 r/min;
wherein the mixed solution is dropwise added at the speed of 0.5-2 mL/min; and
wherein the nano gel solution stands for 8-24 hours.

14. A preparation method of the theaflavin composition capable of promoting wound healing according to claim 13, comprising the following steps:
preparing a sodium acetate buffer solution;
preparing a gelatin buffer solution and a chitosan buffer solution, respectively, with the sodium acetate buffer solution, and filtering the same;
dissolving theaflavin-3-3′-digallate into the chitosan buffer solution to obtain a mixed solution;
dropwise adding the mixed solution into the gelatin buffer solution, stirring, centrifuging, and collecting the precipitate;
redissolving the precipitate into water by 1/10 of the original volume, adding glycerin, propylene glycol, and Carbomer 940 according to a raw material formula, mixing uniformly, and standing to obtain a nano gel solution; and
adding triethanolamine into the nano gel solution, and stirring to obtain the theaflavin composition capable of promoting wound healing;
wherein the gelatin buffer solution and the chitosan buffer solution are filtered using a 0.45 μm aquo-system filter membrane;
wherein the theaflavin-3-3′-digallate is dissolved into the chitosan buffer solution with the aid of ultrasound, and the precipitate is redissolved into water with the aid of ultrasound;
wherein the centrifugation treatment is carried out for 15-60 min at the temperature of 2-6° C. at the rotation speed of 3000-5000 r/min;
wherein the magnetic stirring treatment is specifically conducted for 20-50 min at the rotation speed of 250-350 r/min;
wherein the mixed solution is dropwise added at the speed of 0.5-2 mL/min; and
wherein the nano gel solution stands for 8-24 hours.

15. A method for promoting or accelerating wound healing, comprising applying an effective amount of a theaflavin composition to a wound in need thereof, wherein the theaflavin composition is the theaflavin composition capable of promoting wound healing according to claim 12.

16. A theaflavin composition capable of promoting wound healing, which comprises the theaflavin-3-3′-digallate, glycerin, propylene glycol, triethanolamine, and Carbomer 940;
wherein the theaflavin-3-3′-digallate, glycerin, propylene glycol, triethanolamine, and Carbomer 940 are contained in the theaflavin composition at a mass ratio of 10:19:10:1:1; and,
wherein the theaflavin composition capable of promoting wound healing is prepared by a method comprising the steps of:
(1) dissolving 100 mg of the theaflavin-3-3′-digallate in 10 mL of water with the aid of ultrasound;
(2) adding glycerin, propylene glycol, and Carbomer 940 according to a raw material formula, and standing to obtain a gel solution; and
(3) adding triethanolamine into the gel solution, and stirring to obtain the theaflavin composition capable of promoting wound healing.
